(19) United States
(12) Patent Application Publication
(10) Pub. No.: US 2026/0250581 A1
(43) Pub. Date: Aug. 27, 2026

(54) PROCESSES FOR PREPARING C2 TO C4 HYDROCARBONS USING HYBRID CATALYSTS

(71) Applicants: Dow Global Technologies LLC, Midland, MI (US); Dow Silicones Corporation, Midland, MI (US)

(72) Inventors: Vera Santos, Terneuzen (NL); Glenn Pollefeyt, Terneuzen (NL); Andrzej Malek, Midland, MI (US); David F. Yancey, Midland, MI (US); Ewa A. Tocha-Bielak, Terneuzen (NL); Steven J. Rozeveld, Midland, MI (US); Stuart Leadley, Seneffe (BE); Alexey Kirilin, Terneuzen (NL); Adam Chojecki, Terneuzen (NL)

(73) Assignees: Dow Global Technologies LLC, Midland, MI (US); Dow Silicones Corporation, Midland, MI (US)

(21) Appl. No.: 18/995,527

(22) PCT Filed: Jul. 13, 2023
(86) PCT No.: PCT/US2023/070115

§ 371 Date: Jan. 16, 2025

Related U.S. Application Data
(60) Provisional application No. 63/391,477, filed on Jul. 22, 2022.

(51) Int. Cl. C10G 2/00; B01J 23/08; B01J 23/10; B01J 29/85; B01J 37/00; B01J 37/08; B01J 37/12

(52) U.S. Cl. CPC C10G 2/334; B01J 23/08; B01J 23/10; B01J 29/85; B01J 37/0018; B01J 37/08; B01J 37/12; C10G 2/50; C10G 2300/4006; C10G 2400/20

(57) ABSTRACT

This disclosure relates to processes for preparing C₂ to C₄ hydrocarbons comprising introducing a feed stream comprising hydrogen gas and a carbon-containing gas selected from the group consisting of carbon monoxide, carbon dioxide, and mixtures thereof into a reaction zone of a reactor, and converting the feed stream into a product stream comprising C₂ to C₄ hydrocarbons in the reaction zone in the presence of a hybrid catalyst. The hybrid catalyst comprises a metal oxide catalyst component supported on zirconia and a microporous catalyst component.

### CROSS-REFERENCE TO RELATED APPLICATIONS

This application claims the benefit of U.S. Provisional Application Ser. No. 63/391,477 filed Jul. 22, 2022, the entire disclosure of which is hereby incorporated herein by reference.

### BACKGROUND

### Field

The present disclosure relates to processes that efficiently convert various carbon-containing streams to C₂ to C₄ hydrocarbons. In particular, the present disclosure relates to hybrid catalysts and process methods for converting gas feeds into desirable hydrocarbon products.

### Technical Background

For a number of industrial applications, hydrocarbons are used, or are starting materials used, to produce plastics, fuels, and various downstream chemicals. Such hydrocarbons include C₂ to C₄ materials, such as ethene, propene and butenes (also commonly referred to as ethylene, propylene, and butylenes, respectively). A variety of processes for producing these lower hydrocarbons have been developed, including petroleum cracking and various synthetic processes. However, many of these synthetic processes have low carbon conversion and much of the feed gas either (1) does not get converted and exits the process in the same form as the feed gas; (2) is converted to CO₂; and/or (3) these synthetic processes have low stability over time and the catalyst rapidly loses its activity for carbon conversion to desirable products. For example, many synthetic processes tend to have catalysts with rapidly decreasing activity—and, thus, decreased C₂ to C₄ hydrocarbon production-over time. Accordingly, a need exists for processes that include hybrid catalysts, rather than individual catalytic components that have a high conversion of feed carbon to desired products, such as, for example, C₂ to C₄ hydrocarbons.

### SUMMARY

Embodiments of the present disclosure address these and other needs by preparation of hybrid catalysts and processes using such catalysts. A hybrid catalyst includes a combination of a metal oxide catalyst component, and a microporous catalyst component. The metal oxide catalyst component and the microporous catalyst component can be combined into a single catalyst body to from the hybrid catalyst. The metal oxide component and the microporous catalyst component operate in tandem so that the hybrid catalyst can be used for the direct conversion of a feed stream comprising hydrogen gas and a carbon-containing gas, such as syngas, to C₂ to C₄ hydrocarbons. Further, the metal oxide catalyst component can include both gallium oxide and a rare earth oxide, which can improve the stability of the hybrid catalyst during use in catalytic processes.

According to one or more other aspects of the present disclosure, a process for preparing C₂-C₄ hydrocarbons comprises introducing a feed stream comprising hydrogen gas and a carbon-containing gas selected from the group consisting of carbon monoxide, carbon dioxide, and mixtures thereof into a reaction zone of a reactor; and converting the feed stream into a product stream comprising C₂ to C4 hydrocarbons in the reaction zone in the presence of a hybrid catalyst. The hybrid catalyst comprises a metal oxide catalyst component supported on zirconia, the metal oxide catalyst component comprising gallium oxide and a rare earth oxide; and a microporous catalyst component comprising a molecular sieve having 8 MR (Member Ring) pore openings.

Additional features and advantages will be set forth in the detailed description which follows, and in part will be readily apparent to those skilled in the art from that description or recognized by practicing the embodiments described herein, including the detailed description which follows and the claims.

It is to be understood that both the foregoing general description and the following detailed description describe various embodiments and are intended to provide an overview or framework for understanding the nature and character of the claimed subject matter.

### BRIEF DESCRIPTION OF THE DRAWINGS

FIG. 1 depicts the normalized CO conversion as a function of time on stream using metal oxide catalyst components described herein. FIG. 1A depicts the normalized CO conversion using Comparative Example 1 and Example 1. FIG. 1B depicts the normalized CO conversion using Comparative Example 1 and Example 2. FIG. 1C depicts the normalized CO conversion using Example 1 and Example 3.

FIG. 2 depicts the normalized CO conversion as a function of time on stream using Comparative Example 2 and Example 4, as described herein.

### DETAILED DESCRIPTION

Reference will now be made in detail to embodiments of processes for preparing C₂ to C₄ hydrocarbons from a feed stream comprising hydrogen gas and a carbon-containing gas. As used herein, “a carbon-containing gas” refers to a gas selected from carbon monoxide, carbon dioxide, and mixtures thereof. Conventional methods exhibit a low feed carbon conversion and/or deactivate quickly as they are used, such as, for example, by having an increase in methane production or a decreased syngas conversion rate, which can lead to a low olefin yield and/or low stability for a given set of operating conditions over a given amount of time. In contrast, hybrid catalysts comprising rare earth oxides disclosed herein can exhibit a high and steady yield of C₂ to C₄ hydrocarbons, such as C₂ to C₄ olefins when compared to rare earth oxide-free hybrid catalysts. The preparation and composition of such hybrid catalysts and the process of using such hybrid catalysts are discussed below.

In embodiments, hybrid catalysts can closely couple independent reactions of at least two independent catalysts. In a first step, a feed stream comprising hydrogen gas (H₂) and a carbon-containing gas selected from the group consisting of carbon monoxide (CO), carbon dioxide (CO₂), or a mixture of CO and CO₂, such as, for example, syngas, can be converted into an intermediate(s) such as oxygenated hydrocarbons. In a subsequent step, these intermediates can be converted into a product stream comprising hydrocarbons (mostly short chain hydrocarbons, such as, for example C₂ to C₄ hydrocarbons). The continued formation and consumption of the intermediate oxygenates formed in the first step by the reactions of the second step can reduce or eliminate a thermodynamic limit on conversion.

In embodiments, hybrid catalysts include a metal oxide catalyst component, which can convert the feed stream to oxygenated hydrocarbons, and a microporous catalyst component, which can convert the oxygenated hydrocarbons to hydrocarbons. The metal oxide catalyst component is combined with a microporous catalyst component. It should be understood that, as used herein, the “metal oxide catalyst component” includes metals in various oxidation states. In some embodiments, the metal oxide catalyst component may include more than one metal oxide and individual metal oxides within the metal oxide catalyst component may have different oxidation states. Thus, the metal oxide catalyst component is not limited to comprising metal oxides with homogenous oxidation states.

In embodiments, the metal oxide catalyst component is supported on zirconia (ZrO₂). In embodiments, the zirconia support of the metal oxide catalyst component can be phase pure zirconia. As used herein, “phase pure zirconia” refers to zirconia to which no other materials have intentionally been added during formation. Thus, phase pure zirconia includes zirconia with small amounts of components other than zirconium (including oxides other than zirconia) that are unintentionally present in the zirconia as a natural part of the zirconia formation process, such as, for example, hafnium (Hf). In other embodiments, the zirconia can be non-phase pure zirconia, such as but not limited to zirconia doped with calcium, yttria, lanthanum, cerium or rare earth elements.

In embodiments, the metal oxide catalyst component comprises gallium oxide. As used herein, “gallium oxide” refers to gallium in various oxidation states. In embodiments, gallium oxide can be deposited on the surface of zirconia or be in solid solution with ZrO₂. In embodiments, gallium oxide may include but not be limited to Ga₂O₃, GaO(OH), and Ga₅O₇(OH). Gallium oxide can also include polymorphs of Ga₂O₃, such as monoclinic (β-Ga₂O₃), rhombohedral (α-Ga₂O₃), defective spinel (γ-Ga₂O₃), cubic (δ-Ga₂O₃), or orthorhombic (ε-Ga₂O₃) structures. In other embodiments, gallium oxide may include gallium in more than one oxidation state. For example, individual gallium may be in different oxidation states. Gallium oxide is not limited to comprising gallium in homogenous oxidation states.

In embodiments, the composition of the metal oxide catalyst component is designated by a weight percentage of gallium from the gallium oxide. In embodiments, the weight percentage of gallium can be measured analytically using X-ray fluorescence, assuming a fully oxidic balance. In embodiments, the metal oxide catalyst component can comprise less than or equal to 10 wt. % gallium based on the total weight of the metal oxide catalyst component. For instance, in embodiments, the metal oxide catalyst component comprises gallium in an amount of less than or equal to 9 wt. %, less than or equal to 8 wt. %, less than or equal to 7 wt. %, less than or equal to 6 wt. %, or even less than or equal to 5 wt. %, based on the total weight of the metal oxide catalyst component. In embodiments, the metal oxide catalyst component comprises gallium in an amount from 1 wt. % to 10 wt. %, from 1 wt. % to 9 wt. %, from 1 wt. % to 8 wt. %, from 1 wt. % to 7 wt. %, from 1 wt. % to 6 wt. %, from 1 wt. % to 5 wt. %, from 1 wt. % to 4 wt. %, from 1 wt. % to 3 wt. %, from 2 wt. % to 10 wt. %, from 2 wt. % to 9 wt. %, from 2 wt. % to 8 wt. %, from 2 wt. % to 7 wt. %, from 2 wt. % to 6 wt. %, from 2 wt. % to 5 wt. %, from 2 wt. % to 4 wt. %, or from 2 wt. % to 3 wt. % based on the total weight of the metal oxide catalyst component. Without being bound by any particular theory, it is believed that while zirconia or gallium oxide admixed with a microporous solid in a hybrid catalyst may convert carbon-containing components to C₂ to C₄ hydrocarbons, it is believed that the gallium oxide and the zirconia synergistically improve yield for C₂ to C₄ hydrocarbons.

In embodiments, the metal oxide catalyst component comprises a rare earth oxide. As used herein, “rare earth oxide” refers to an oxide comprising a rare earth element, which includes scandium, yttrium, and elements having an atomic number from 57 to 71, such as but not limited to, samarium, gadolinium, dysprosium, lanthanum, cerium, and neodymium.

In embodiments, the composition of the metal oxide catalyst component is designated by a weight percentage of rare earth elements from the rare earth oxide. In embodiments, the weight percentage of rare earth elements can be measured analytically using X-ray fluorescence, assuming a fully oxidic balance. In embodiments, the metal oxide catalyst component can comprise less than or equal to 7 wt. % rare earth elements based on the total weight of the metal oxide catalyst component. For instance, in embodiments, the metal oxide catalyst component comprises rare earth elements in an amount of less than or equal to 6 wt. %, or even less than or equal to 5 wt. %, based on the total weight of the metal oxide catalyst component. In embodiments, the metal oxide catalyst component comprises rare earth elements in an amount greater than 1 wt. %, greater than 2 wt. %, or even greater than 3 wt. %. In embodiments, the metal oxide catalyst component comprises rare earth elements in an amount from 1 wt. % to 7 wt. %, from 1 wt. % to 6 wt. %, from 1 wt. % to 5 wt. %, from 1 wt. % to 4 wt. %, from 2 wt. % to 7 wt. %, from 2 wt. % to 6 wt. %, from 2 wt. % to 5 wt. %, or from 2 wt. % to 4 wt. %, based on the total weight of the metal oxide catalyst component. Without being bound by any particular theory, it is believed that the addition of rare earth oxides to the metal oxide catalyst component may decrease a deactivation rate of the hybrid catalyst comprising the metal oxide catalyst component catalytic reactions, such as the conversion of carbon-containing gases to C₂-C₄ hydrocarbons. It is further believed the addition of rare earth oxides to the metal oxide catalyst component can increase the stability of the metal oxide catalyst component during catalytic reactions. It is believed that less than 1 wt. % of rare earth elements in the metal oxide catalyst component may not provide sufficient reduction in the deactivation rate of the hybrid catalyst comprising the metal oxide catalyst component. Further, it is believed that greater than 7 wt. % of rare earth elements in the metal oxide catalyst component may decrease catalytic activity of the hybrid catalyst.

In other instances, additional rare earth elements and/or transition metals can be co-deposited with gallium precursor or introduced only when the mixed composition including gallium oxide and zirconia has been prepared in the first place. For instance, in embodiments, the metal oxide catalyst component can further comprise transition metal elements such as nickel (Ni), palladium (Pd), or platinum (Pt).

In view of the above, one method for making the metal oxide catalyst component of the hybrid catalyst is by incipient wetness impregnation. In such a method, an aqueous mixture of a gallium precursor material, such as gallium(III) nitrate hydrate (Ga(NO₃)₃·xH₂O) and an aqueous mixture of a rare earth element precursor material, such as lanthanum(III) nitrate hexahydrate (La(NO₃)₃·6H₂O) are added to zirconia, such as zirconia powder or solution, to form a mixture. The amounts of each material in the mixture can be controlled based on a desired ratio of the gallium to zirconia, rare earth element to zirconia, or gallium to rare earth element. In other embodiments, the gallium oxide and/or rare earth oxide may be deposited or distributed on the zirconia oxide by chemical vapor deposition (CVD) method. However, the method for making the metal oxide catalyst component of the hybrid catalyst is not particularly limited and any method that can apply a fine layer of gallium oxide and rare earth oxide on the surface of zirconium oxide can be used according to embodiments. It should be understood that the total amount of gallium precursor and rare earth element precursor that are mixed with the zirconia will be determined on the desired target amount of gallium and rare earth element in the metal oxide catalyst component.

As discussed previously, the metal oxide catalyst component can be supported on zirconia. In embodiments, the zirconia can include zirconia particles having a crystalline structure. In embodiments, the zirconia include zirconia particles having a monoclinic structure. In one or more embodiments, the zirconia consists essentially of or consists of crystalline zirconia particles. In embodiments, the zirconia consist essentially of or consist of monoclinic zirconia particles. According to some embodiments, the zirconia has a BET surface area that is greater than or equal to 5 meters squared per gram (m²/g), such as greater than 10 m²/g, greater than 20 m²/g, greater than 30 m²/g, greater than 40 m²/g, greater than 50 m²/g, greater than 60 m²/g, greater than 70 m²/g, greater than 80 m²/g, greater than 90 m²/g, greater than 100 m²/g, greater than 110 m²/g, greater than 120 m²/g, greater than 130 m²/g, or greater than 140 m²/g. According to some embodiments, the maximum BET surface area of the zirconia is 150 m²/g. Accordingly, in some embodiments, the BET surface area of the zirconia is from 5 m²/g to 150 m²/g, from 10 m²/g to 150 m²/g, from 20 m²/g to 150 m²/g, such as from 30 m²/g to 150 m²/g, from 40 m²/g to 150 m²/g, from 50 m²/g to 150 m²/g, from 60 m²/g to 150 m²/g, from 70 m²/g to 150 m²/g, from 80 m²/g to 150 m²/g, from 90 m²/g to 150 m²/g, from 100 m²/g to 150 m²/g, from 110 m²/g to 150 m²/g, from 120 m²/g to 150 m²/g, from 130 m²/g to 150 m²/g, or from 140 m²/g to 150 m²/g. In some embodiments, the BET surface area of the zirconia is from 5 m²/g to 140 m²/g, such as from 5 m²/g to 130 m²/g, from 5 m²/g to 120 m²/g, from 5 m²/g to 110 m²/g, from 5 m²/g to 100 m²/g, from 5 m²/g to 90 m²/g, from 5 m²/g to 80 m²/g, from 5 m²/g to 70 m²/g, from 5 m²/g to 60 m²/g, from 5 m²/g to 50 m²/g, from 5 m²/g to 40 m²/g, from 5 m²/g to 30 m²/g, from 5 m²/g to 20 m²/g, or from 5 m²/g to 10 m²/g. In some embodiments, the BET surface area of the zirconia is from 10 m²/g to 140 m²/g, from 20 m²/g to 130 m²/g, from 30 m²/g to 120 m²/g, from 40 m²/g to 110 m²/g, from 50 m²/g to 100 m²/g, from 60 m²/g to 90 m²/g, or from 70 m²/g to 80 m²/g.

In embodiments, once the gallium precursor material, the rare earth element precursor material, and zirconia are adequately mixed, the metal oxide catalyst component may be dried at temperatures less than 200 degrees Celsius (° C.), such as less than 175° C., less than 150° C., less than 100° C., or about 85° C. Subsequent to the drying, the metal oxide catalyst component may be calcined at temperatures from 400° C. to 800° C., such as from 400° C. to 775° C., from 400° C. to 750° C., from 400° C. to 725° C., from 400° C. to 700° C., from 400° C. to 675° C., from 400° C. to 650° C., from 400° C. to 625° C., from 400° C. to 600° C., from 500° C. to 800° C., from 500° C. to 775° C., from 500° C. to 750° C., from 500° C. to 725° C., from 500° C. to 700° C., from 500° C. to 675° C., from 500° C. to 650° C., from 550° C. to 800° C., from 550° C. to 775° C., from 550° C. to 750° C., from 550° C. to 725° C., from 550° C. to 700° C., from 550° C. to 675° C., from 550° C. to 650° C., from 600° C. to 800° C., from 600° C. to 775° C., from 600° C. to 750° C., from 600° C. to 725° C., from 600° C. to 700° C., from 600° C. to 675° C., from 600° C. to 650° C., from 650° C. to 800° C., from 650° C. to 775° C., from 650° C. to 750° C., from 650° C. to 725° C., from 650° C. to 700° C., or from 700° C. to 800° C. After calcining, the composition of the mixed metal oxide catalyst component is determined and may be reported as a weight of gallium and a weight of the rare earth element as disclosed herein.

Without being bound by any particular theory, it is believed that calcining the metal oxide catalyst component at a higher temperature may result in improved catalytic stability of the metal oxide catalyst component and/or the hybrid catalyst comprising the metal oxide catalyst component during catalytic reactions, such as the conversion of carbon-containing gases to C₂-C₄-hydrocarbons. Higher calcination temperatures can reduce the BET surface area of the metal oxide catalyst component, which can reduce catalytic activity. However, the inclusion of a rare earth element in the metal oxide catalyst component can allow for the metal oxide catalyst component to retain a higher BET surface area, even when calcining at higher temperatures. It is believed that calcining the metal oxide catalyst component at a temperature greater than 800° C. may result in a greater loss of BET surface area of the catalyst, which may decrease the overall performance of the catalyst. It is believed that calcining the metal oxide catalyst component at a temperature below 400° C. may cause structural change in the hybrid catalyst during use in catalytic operation.

It should be understood that according to embodiments, the metal oxide catalyst component may be made by other methods that eventually lead to intimate contact and/or dispersion between the gallium precursor, the rare earth element precursor, and zirconia. Some non-limiting instances include vapor phase deposition of Ga-containing precursors and/or rare earth element-containing precursors (either organic or inorganic in nature), followed by their controlled decomposition. Similarly, processes for dispersing liquid gallium metal can be amended by those skilled in the art to lead to intimate contact between the gallium precursor, rare earth element precursor, and zirconia.

In embodiments, after the metal oxide catalyst component has been prepared—such as, for example, by the methods disclosed above—the metal oxide catalyst component can be mixed with a microporous catalyst component, and a binder to form a single catalyst. In embodiments, the microporous catalyst component can be selected from molecular sieves having 8-MR pore openings and having a framework type selected from the group consisting of the following framework types CHA, AEI, AFX, ERI, LEV, LTA, UFI, RTH, EDI, GIS, MER, RHO, and combinations thereof, the framework types corresponding to the naming convention of the International Zeolite Association. It should be understood that in embodiments, both aluminosilicate and silicoaluminophosphate frameworks may be used. Some embodiments may include tetrahedral aluminosilicates, ALPOs (such as, for example, tetrahedral aluminophosphates), SAPOs (such as, for example, tetrahedral silicoaluminophosphates), and silica-only based tectosilicates. In certain embodiments, the microporous catalyst component may be silicoaluminophosphate having a Chabazite (CHA) framework type. Examples of these may include, but are not necessarily limited to: CHA embodiments selected from SAPO-34 and SSZ-13; and AEI embodiments such as SAPO-18 and SSZ-13. Combinations of microporous catalyst components having any of the above framework types may also be employed. It should be understood that the microporous catalyst component may have different membered ring pore opening depending on the desired product. For instance, microporous catalyst component having 8-MR to 12-MR pore openings could be used depending on the desired product. However, to produce C₂ to C₄ hydrocarbons, a microporous catalyst component having 8-MR pore openings can be used in embodiments.

The metal oxide catalyst component and the microporous catalyst component may be combined with a mass ratio of from 1:10 to 10:1, from 1:10 to 9:1, from 1:10 to 8:1, from 1:10 to 5:1, from 1:10 to 4:1, from 1:10 to 3:1, from 1:8 to 8:1, from 1:8 to 7:1, from 1:8 to 6:1, from 1:8 to 5:1, from 1:8 to 4:1, from 1:5 to 8:1, from 1:5 to 7:1, from 1:5 to 6:1, or from 1:5 to 5:1.

In embodiments, a binder may be added to the mixture of the metal oxide catalyst component and the microporous catalyst component to produce a paste. The binder may be capable of holding the metal oxide catalyst component and the microporous catalyst component together. The paste may be extruded to produce the hybrid catalyst. The hybrid catalyst may be by any suitable extrusion process.

Various binders are considered suitable. For example, the binder may include alumina, zirconia, or both. In embodiments, the binder may include pure alumina. In embodiments, the binder may include pure zirconia. When the binder includes alumina, the alumina binder may be a hydrous alumina. A hydrous alumina composition may be prepared from boehemitic precursors with water and peptizing agent. The binder may be mixed with the metal oxide catalyst component and the microporous catalyst component. After mixing the binder with the metal oxide catalyst component and the microporous catalyst component, the mixture may be extruded, dried, and calcined. After calcination, the binder may form aluminum oxide and bind the metal oxide catalyst component and the microporous catalyst component together to provide mechanical strength to extrude the hybrid catalyst. In embodiments, the binder is a colloidal solution, suspension, or gel of a binder precursor. The binder precursor may include oxides or hydroxides of aluminum, oxides or hydroxides of zirconium, or mixtures thereof. In one embodiment, the binder precursor may include pure alumina, (pseudo) boehmite or gibbsite, or mixtures thereof. In other embodiments, the binder precursor may include pure zirconia, hydrous zirconia, or mixtures thereof.

The metal oxide catalyst component and the microporous catalyst component of the hybrid catalyst may be mixed together by any suitable means to achieve homogenous mixing of all the components prior to extrusion. The metal oxide catalyst component and the microporous catalyst component can be initially mixed as powders to achieve homogeneity in suitable dry mixer, such as a ribbon or plow mixer. The peptized binder precursor can be added to the mixture of the metal oxide catalyst component and the microporous catalyst component and mixed in a suitable heavy duty industrial mixer capable of handling thick paste formulations. Alternatively, the dry pre-mixed metal oxide catalyst component and the microporous catalyst component can be fed directly into the feeding screws of a screw extruder along with the peptized binder precursor composition and mixed directly in the screw extruder. The hybrid catalyst may be extruded into a desired shape by any suitable extrusion method. Examples of shapes include pellets, spherical, or near-spherical. In embodiments, the metal oxide catalyst component may include from 1.0 weight percent (wt. %) to 85.0 wt % of the hybrid catalyst, such as from 5.0 wt % to 80.0 wt %, from 10.0 wt % to 80.0 wt %, from 15.0 wt % to 80.0 wt %, from 20.0 wt % to 80.0 wt %, from 25.0 wt % to 80.0 wt %, from 30.0 wt % to 80.0 wt %, from 35.0 wt % to 80.0 wt %, from 40.0 wt % to 80.0 wt %, from 45.0 wt % to 80.0 wt %, from 50.0 wt % to 80.0 wt %, from 55.0 wt % to 80.0 wt %, from 60.0 wt % to 80.0 wt %, from 65.0 wt % to 80.0 wt %, from 70.0 wt % to 80.0 wt %, or from 75.0 wt % to 80.0 wt %. In some embodiments, the metal oxide catalyst component includes from 1.0 wt % to 80.0 wt %, from 1.0 wt % to 75.0 wt %, from 1.0 wt % to 70.0 wt %, from 1.0 wt % to 65.0 wt %, from 1.0 wt % to 60.0 wt %, from 1.0 wt % to 55.0 wt %, from 1.0 wt % to 50.0 wt %, from 1.0 wt % to 45.0 wt %, from 1.0 wt % to 40.0 wt %, from 1.0 wt % to 35.0 wt %, from 1.0 wt % to 30.0 wt %, from 1.0 wt % to 25.0 wt %, from 1.0 wt % to 20.0 wt %, from 1.0 wt % to 15.0 wt %, from 1.0 wt % to 10.0 wt %, or from 1.0 wt % to 5.0 wt %. In some embodiments, the metal oxide catalyst component includes from 5.0 wt % to 80.0 wt % of the hybrid catalyst, such as from 10.0 wt % to 80.0 wt %, from 15.0 wt % to 80.0 wt %, from 20.0 wt % to 80.0 wt %, from 25.0 wt % to 75.0 wt %, from 30.0 wt % to 70.0 wt %, from 35.0 wt % to 65.0 wt %, from 40.0 wt % to 60.0 wt %, or from 45.0 wt % to 55.0 wt %. In some embodiments, the metal oxide catalyst component includes from 50.0 wt % to 80.0 wt % of the hybrid catalyst, such as from 50.0 wt % to 75.0 wt %, from 50.0 wt % to 70.0 wt %, from 60.0 wt % to 80.0 wt %, from 60.0 wt % to 75.0 wt %, or from 60.0 wt % to 70.0 wt %.

In other embodiments, the metal oxide catalyst component and the microporous catalyst component of the hybrid catalyst may be mixed together by any suitable means available to one skilled in the art, including but not limited to, physical mixing of individual catalysts, bifunctional catalyst pills, and bifunctional catalyst extrudates.

In embodiments, the binder can combine the metal oxide catalyst component and the microporous catalyst component into a single catalyst body, which can improve C₂ to C₄ hydrocarbon yields and carbon conversion compared to a physical mixture comprising individually the metal oxide catalyst component and the microporous catalyst component.

In embodiments, the binder may be a colloidal solution, suspension, or gel of a binder precursor. The binder precursor may include oxides or hydroxides of aluminum, oxides or hydroxides of zirconium, or mixtures thereof. In one embodiment, the binder precursor may include pure alumina, (pseudo) boehmite or gibbsite, or mixtures thereof. In other embodiments, the binder precursor may include pure zirconia, hydrous zirconia, or mixtures thereof. In embodiments where the binder includes hydrous alumina, e.g. pseudo-boehmite or gibbsite, acid is typically added as peptization agent. The peptization ratio is defined as the moles of acid added during peptization over the moles of aluminum, ([H⁺]/[Al]). In one or more embodiments, the peptization ratio may have [H⁺]/[Al] ratio of from 0.005 to 0.1, from 0.01 to 0.1, or about 0.05.

Without being bound by any particular theory, the use of templated molecular sieves (e.g. uncalcined) for the formulation has been found to have a positive impact on the catalyst performance and structural properties, particularly when strongly acidic conditions, such as an [H⁺]/[Al] ratio of more than 0.05, or more than 0.025, are used during the formulation procedure of hybrid catalysts.

In alternative embodiments, the zirconia support may be first mixed with the microporous component and the binder to obtain a hybrid catalyst support. Alternatively, the zirconia support might be generated in-situ during calcination of the hybrid catalyst support. Active metal oxide catalyst components (e.g. gallium, lanthanide and/or transition metal) may be added subsequently to the hybrid catalyst support to achieve a hybrid catalyst by any means known to one skilled in the art, as described in detail above.

In some embodiments, the metal oxide catalyst component and/or the binder are substantially free of silicon and phosphorus. The term “substantially free” of a constituent refers less than 0.5 weight percent (wt. %) of that component in a composition. For example, the mixed metal oxide catalyst component and binder that are substantially free of silicon and phosphorus may have less than 0.5 wt. % silicon and phosphorus based on the combined weight of the mixed metal oxide and binder. Without being bound by any particular theory, it is believed that the reduction of silicon and phosphorus in the metal oxide catalyst component and/or the binder can increase the catalytic performance of the hybrid catalyst.

The hybrid catalyst may be used in methods for converting carbon in a carbon-containing feed stream to C₂ to C₄ hydrocarbons. Such processes will be described in more detail below.

A process for preparing C₂ to C₄ hydrocarbons comprising the hybrid catalyst includes introducing a feed stream including hydrogen gas and a carbon-containing gas selected from the group consisting of carbon monoxide, carbon dioxide, and mixtures thereof into a reaction zone of a reactor, and converting the feed stream into a product stream including C₂ to C₄ hydrocarbons in the reaction zone in the presence of a hybrid catalyst.

According to embodiments, a feed stream is fed into a reaction zone, the feed stream comprising hydrogen (H₂) gas and a carbon-containing gas selected from carbon monoxide (CO), carbon dioxide (CO₂), and combinations thereof. In some embodiments, the H₂ gas is present in the feed stream in an amount of from 10 volume percent (vol %) to 90 vol %, based on combined volumes of the H₂ gas and the gas selected from CO, CO₂, and combinations thereof. The feed stream is contacted with a hybrid catalyst as disclosed and described herein in the reaction zone. The hybrid catalyst includes a metal oxide catalyst component supported on zirconia. The metal oxide catalyst component comprises gallium oxide, and a rare earth oxide The hybrid catalyst can also comprise a microporous catalyst component comprising a molecular sieve have 8-MR (Member Ring) pore openings.

It should be understood that the activity of the hybrid catalyst will be higher for feed streams containing CO as the carbon-containing gas, and that the activity of the hybrid catalyst decreases as a larger portion of the carbon-containing gas in the feed stream is CO₂. However, that is not to say that the hybrid catalyst disclosed and described herein cannot be used in methods where the feed stream includes CO₂ as all, or a large portion, of the carbon-containing gas.

The feed stream is contacted with the hybrid catalyst in the reaction zone under reaction conditions sufficient to form a product stream comprising C₂ to C₄ hydrocarbons. The reaction conditions include a temperature within the reaction zone ranging, according to one or more embodiments, from 350° C. to 480° C., from 375° C. to 450° C., from 400° C. to 450° C., from 350° C. to 425° C., from 375° C. to 425° C., from 400° C. to 425° C., from 350° C. to 400° C., or from 375° C. to 400° C.

In embodiments, the reaction conditions include a pressure inside the reaction zone of at least 1 bar (100 kilopascals (kPa), such as at least 5 bar (500 kPa), at least 10 bar (1,000 kPa), at least 15 bar (1,500 kPa), at least 20 bar (2,000 kPa), at least 25 bar (2,500 kPa), at least 30 bar (3,000 kPa), at least 35 bar (3,500 kPa), at least 40 bar (4,000 kPa), at least 45 bar (4,500 kPa), at least 50 bar (5,000 kPa), at least 55 bar (5,500 kPa), at least 60 bar (6,000 kPa), at least 65 bar (6,500 kPa), at least 70 bar (7,000 kPa), at least 75 bar (7,500 kPa), at least 80 bar (8,000 kPa), at least 85 bar (8,500 kPa), at least 90 bar (9,000 kPa), at least 95 bar (9,500 kPa), or at least 100 bar (10,000 kPa). In other embodiments, the reaction conditions include a pressure inside the reaction zone is from 5 bar (500 kPa) to 100 bar (10,000 kPa), such as from 10 bar (1,000 kPa) to 95 bar (9,500 kPa), from 15 bar (1,500 kPa) to 90 bar (9,000 kPa), from 20 bar (2,000 kPa) to 85 bar (8,500 kPa), from 25 bar (2,500 kPa) to 80 bar (8,000 kPa), from 30 bar (3,000 kPa) to 75 bar (7,500 kPa), from 35 bar (3,500 kPa) to 70 bar (7,000 kPa), from 40 bar (4,000 kPa) to 65 bar (6,500 kPa), from 45 bar (4,500 kPa) to 60 bar (6,000 kPa), or from 50 bar (5,000 kPa) to 55 bar (5,500 kPa). In some embodiments, the pressure inside the reaction zone is from 20 bar (2,000 kPa) to 60 bar (6,000 kPa).

According to embodiments, the gas hourly space velocity (GHSV) within the reaction zone is from 500 per hour (/h) to 12,000/h, such as from 500/h to 10,000/h, from 1,200/h to 12,000/h, from 1,500/h to 10,000/h, from 2,000/h to 9,500/h, from 2,500/h to 9,000/h, from 3,000/h to 8,500/h, from 3,500/h to 8,000/h, from 4,000/h to 7,500/h, from 4,500/h to 7,000/h, from 5,000/h to 6,500/h, or from 5,500/h to 6,000/h. In some embodiments the GHSV within the reaction zone is from 1,800/h to 3,600/h, such as from 2,000/h to 3,600/h, from 2,200/h to 3,600/h, from 2,400/h to 3,600/h, from 2,600/h to 3,600/h, from 2,800/h to 3,600/h, from 3,000/h to 3,600/h, from 3,200/h to 3,600/h, or from 3,400/h to 3,600/h. In some embodiments, the GHSV within the reaction zone is from 1,800/h to 3,400/h, such as from 1,800/h to 3,200/h, from 1,800/h to 3,000/h, from 1,800/h to 2,800/h, from 1,800/h to 2,600/h, from 1,800/h to 2,400/h, from 1,800/h to 2,200/h, or from 1,800/h to 2,000/h. In some embodiments, the GHSV within the reaction is from 2,000/h to 3,400/h, such as from 2,200/h to 3,200/h, from 2,400/h to 3,000/h, or from 2,600/h to 2,800/h.

In embodiments, using hybrid catalysts disclosed and described herein along with the process conditions disclosed and described herein, the process stability and/or productivity of the hybrid catalysts can be improved by maintaining a high carbon conversion rate. Within the process ranges disclosed, the conversion of the feed containing carbon oxides and hydrogen can be carried out in a series of rectors with an intermediate knock-out of water by-product by the means of e.g., phase separation, membrane separation, or some type of water-selective absorptive or adsorptive process. Further directing the partially converted and water-free effluent to the subsequent reactor in series and repeating this manner of technological operations will have an overall effect of enhancing the olefin yield.

In embodiments, using hybrid catalysts disclosed and described herein along with the process conditions disclosed and described herein, the process may have C₂-C₃ olefin selectivity/paraffin selectivity ratio of greater than or equal to 2, from 2 to 20, from 2 to 10, from 2 to 8, from 2 to 6, from 3 to 11, from 3 to 10, from 3 to 8, from 3 to 6, or about 4.

### EXAMPLES

The following examples illustrate features of the present disclosure but are not intended to limit the scope of the disclosure. For each of the following examples and comparative examples, the microporous catalyst component was prepared as follows: SAPO-34 was synthesized per literature procedures (Lok, B. M.; Messina, C. A.; Patton, R. L.; Gajek, R. T.; Cannan, T. R.; Flanigen, E. M. Crystalline silicoaluminophosphates. U.S. Pat. No. 4,440,871A, 1984). When using calcined SAPO-34, the materials was calcined in air using the following program: 25° C. raise to 600° C. at a heating rate of 2° C./min, hold at 600° C. for 4 hours (h), cool down to 25° C. in 4 h.

### Example 1

A metal oxide catalyst component comprising gallium and lanthanum on a zirconia support was prepared by an incipient wetness impregnation method. An impregnation solution of gallium(III) nitrate hydrate (Ga(NO₃)₃·xH₂O) and lanthanum(III) nitrate hexahydrate (La(NO₃)₃·6H₂O) with a concentration of respectively 0.86 mol/L and 0.86 mol/L in DI water was prepared. 2 g of 40 mesh (400 μm) to 80 mesh (177 μm) ZrO₂ support (manufactured by NORPRO, product code SZ31164, BET surface area=100 m²/g, more than 95% monoclinic phase by XRD, pore volume=0.4 mL/g measured by DI water) was weighed and placed into a glass vial. After that, 0.8 mL of the Ga and La impregnation solution was added dropwise to the support while constantly shaking. After impregnation, the metal oxide catalyst component was dried at 80° C. in a forced convection oven overnight and calcined in a muffle furnace using the following program: from 25° C. to 550° C. at a heating rate of 3° C./min held at 550° C. for 4 hours. After calcination, the catalyst was re-sieved to 40-80 mesh.

### Example 2

A metal oxide catalyst component comprising gallium on a zirconia support was prepared by an incipient wetness impregnation method. An impregnation solution of gallium(III) nitrate hydrate (Ga(NO₃)₃·xH₂O) with a concentration of 0.86 mol/L in DI water was prepared. 2 g of 40 mesh (400 μm) to 80 mesh (177 μm) ZrO₂ support (manufactured by NORPRO, product code SZ31164, BET surface area=100 m²/g, more than 95% monoclinic phase by XRD, pore volume=0.4 mL/g measured by DI water) was weighed and placed into a glass vial. After that, 0.8 mL of the Ga impregnation solution was added dropwise to the support while constantly shaking. After impregnation, the metal oxide catalyst component was dried at 80° C. in a forced convection oven for 12 hours and calcined in a muffle furnace using the following program: from 25° C. to 700° C. at a heating rate of 3° C./min held at 700° C. for 4 hours. After calcination, the catalyst was re-sieved to 40-80 mesh.

### Example 3

The metal oxide catalyst component was prepared according to Example 1, but was calcined from 25° C. to 700° C. at a heating rate of 3° C./min held at 700° C. for 4 hours.

### Example 4

A metal oxide catalyst component comprising gallium and lanthanum on a zirconia support was prepared by an incipient wetness impregnation method. An impregnation solution of gallium(III) nitrate hydrate (Ga(NO₃)₃·xH₂O) and lanthanum(III) nitrate hexahydrate (La(NO₃)₃·6H₂O) with a concentration of respectively 0.73 mol/L and 0.22 mol/L in DI water was prepared. 10 g of ZrO₂ support (manufactured by Daiichi Kigenso Kagaku-Kogyo CO., LTD. (DKKK), product code Z3186, BET surface area=85 m²/g, more than 95% monoclinic phase by XRD, pore volume=0.55 mL/g measured by DI water) was weighed and placed into a glass vial. After that, 5.5 mL of the Ga and La impregnation solution was added dropwise to the support while constantly shaking. After impregnation, the metal oxide catalyst component was dried at 85° C. in a forced convection oven for 12 hours and calcined in a muffle furnace using the following program: from 25° C. to 700° C. at a heating rate of 3° C./min held at 700° C. for 4 hours. After calcination the catalyst was re-sieved to smaller than 200 mesh size (smaller than 75 μm) to remove larger agglomerated particles.

A powder was prepared by mixing 8.0 g of the metal oxide catalyst component described above with 1.808 g of uncalcined SAPO-34 (smaller than 200 mesh size, smaller than 75 μm) for 10 min using a mortar and pestle. Separately, pseudoboehmite (AlOOH) (manufactured by Sasol, tradename Catapal D) was peptized in water using HNO₃ (65 wt. % in H₂O) at a HNO₃/Al ratio of 0.05, and a total solid content of 35 wt. %. The peptized pseudoboehmite mixture was added to the dried powders to form a paste, targeting a pseudoboehmite concentration of 24.6 wt. % on total solids basis (Catapal D, SAPO-34 and Metal Oxide). The paste was subsequently mixed for at least 10 minutes using the mortar and pestle until an extrudable paste was obtained. The paste was transferred to a ceramic dish and dried at 85° C. for 12 hours to form a dried precursor. The dried precursor was heated from 25° C. to 600° C. at a heating rate of 2° C./min in a static muffle furnace and held at 600° C. for 4 hours to form a hybrid catalyst. After calcination, the hybrid catalyst was crushed and sieved to 40 mesh (400 μm) to 80 mesh (177 μm) for testing.

### Comparative Example 1

The metal oxide catalyst component was prepared according to Example 2, but was calcined from 25° C. to 550° C. at a heating rate of 3° C./min held at 550° C. for 4 hours.

### Comparative Example 2

The hybrid catalyst was prepared according to Example 4, but the metal oxide catalyst component was calcined from 25° C. to 550° C. at a heating rate of 3° C./min held at 550° C. for 4 hours.

### Example 5—Catalyst Composition Data

The weight percentages of gallium and lanthanum in oxide forms, assuming oxygen balance, of the metal oxide catalyst components in Examples 1, 2, 3, and Comparative Example 1 were determined by X-ray fluorescence.) and are reported in Table 1. Additionally, the calcination temperature, weight percentage of tetragonal zirconia (t-ZrO₂) relative to the total weight of zirconia, and the BET surface area of Examples 1-3 and Comparative Example 1 are also reported in Table 1. The t-ZrO₂ wt. % was approximated by whole pattern fitting of X-Ray Diffraction data using JADE Professional software, which uses the most stable oxide soichiometry. The BET surface area was calculated by N₂ physisorption using at Micromeritics Tristar 3020. Prior to the BET measurement, each sample was pretreated under nitrogen at 200° C. for 2 h using a Degassing Module SmartPrep 065 (Micromeritics Instrument Corporation). A typical adsorption/desorption isotherm was recorded at 77.4 K using nitrogen as adsorbate with a molecular cross-section area of 0.162 nm². The data is processed in a region where the range of partial pressure is 0.05<p/p0<0.3 using a macro-based Excel tool to linearly fit with an R-squared value of >0.999. For the determination of the pore volume and pore distributions the adsorption and desorption isotherm (ranging from 0.05 up to 1 partial pressure (p/p0) and back to 0.05 was measured with the same adsorbate.

TABLE 1

| | | | Calcination | | |
|---|---|---|---|---|---|
| | Ga, | La, | Temperature | | |
| Catalyst | wt. % | wt. % | ° C. | t-ZrO₂, % | BET, m²/g |
| Example 1 | 2.74 | 3.89 | 550 | 10.0 | 82.57 |
| Example 2 | 2.99 | 0 | 700 | 7.50 | 44.60 |
| Example 3 | 2.79 | 3.99 | 700 | 10.3 | 70.80 |
| Comparative | 2.90 | 0 | 550 | 8.50 | 68.77 |
| Example 1 | | | | | |

As shown in Table 1, the comparison of Example 2 versus Comparative Example 1 demonstrates that calcining the metal oxide catalyst component at a higher temperature results in a decrease in the BET surface area from 68.77 to 44.60 m²/g. The comparison of Example 1 versus Comparative Example 1 demonstrates that the addition of lanthanum in the metal oxide catalyst component results in an increase of the BET surface area from 68.77 to 82.57 m²/g. Further, the comparison of Example 3 and Example 1 demonstrates that calcining the metal oxide catalyst component comprising lanthanum at a higher temperature results in a modest reduction of the BET surface area from 82.57 to 70.80 m²/g. Thus, the inclusion of the rare earth oxide in the metal oxide catalyst component can enable improved stability with higher calcination temperatures while only moderately affecting the BET surface area in comparison to metal oxide catalyst components that do not include the rare earth oxide.

### Example 6—Catalytic Performance Data

The mixed metal oxide catalysts described in Examples 1 to 3 and Comparative Example 1 were physically mixed with calcined SAPO-34 in 40-80 mesh sieve fraction in a 4:1 mass ratio prior to testing. Testing of the hybrid catalysts were performed in a stainless steel fixed bed reactor system (7.7 mm internal diameter, 38.4 cm length) under the following conditions: 420° C., 40 bar and a H₂/CO ratio of approximately 3. These experiments were conducted at iso-conversion levels with a CO conversion of approximately 65%. The volume of balance gas was 10%.

Prior to contacting with syngas, the catalyst was heated under nitrogen (N₂) to reaction temperature and pressure. The reactor effluent composition was obtained by gas chromatography and the conversion and carbon based selectivities are calculated using the following equations:

$${WHSV}_{( MMO + SAPO - 34 )} = ( {F}_{CO} + {F}_{H 2} ) / ( {W}_{MMO} + {W}_{SAPO - 34} ) \qquad ( 1 )$$

$$WHSV = ( {F}_{CO} + {F}_{H 2} ) / {W}_{Catalyst} \qquad ( 2 )$$

where F_{CO} and F_{H2} are defined as the mass flow rates of CO and H₂ respectively, and W_{MMO}, W_{SAPO-34} and W_{catalyst} are defined as the mass of the metal oxide catalyst component, mass of SAPO-34 component and total catalyst mass (including binder), respectively.

$${X}_{CO} ( \% ) = ( {F}_{CO , in} - {F}_{CO , out} ) / {F}_{CO , in} ] \cdot 100 ; \qquad ( 3 )$$

$$and$$

$${S}_{j} ( \% ) = [ {α}_{j} \cdot {F}_{j , out} / ( {F}_{CO , in} - {F}_{CO , out} ) ] \cdot 100 , \qquad ( 4 )$$

where X_{CO} is defined as the CO conversion (%), F_{CO}, in is defined as the molar inlet flow of CO (μmol/s), F_{CO, out} is the molar outlet flow of CO (μmol/s), Sⱼ is defined as the carbon based selectivity to product j (%), αⱼ the number of carbon atoms for product j, F_{j, out} is the molar outlet flow of product j (μmol/s). All data was collected under steady state conditions, after at least 40 hours time on stream.

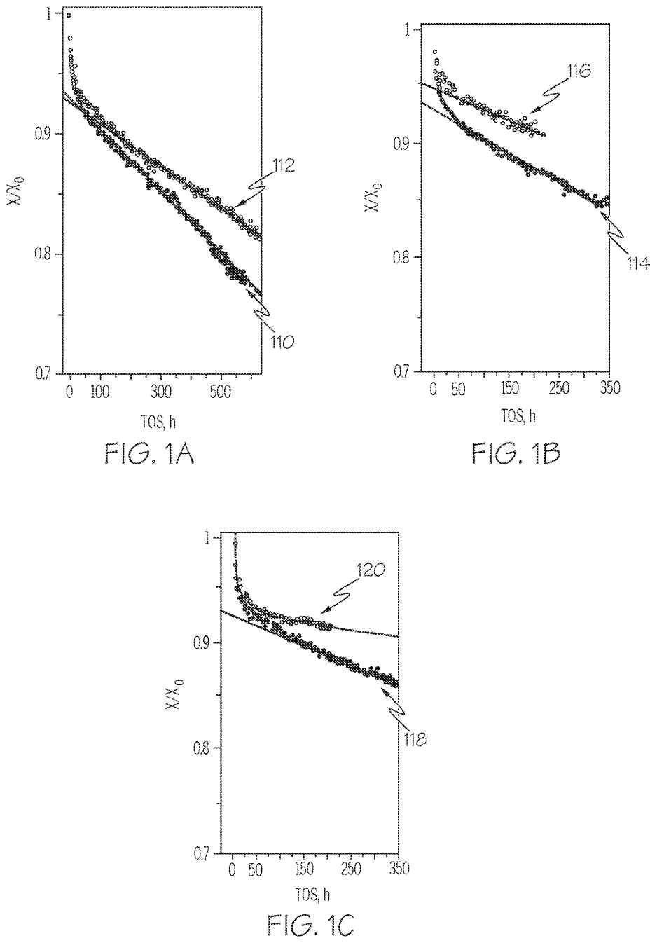

The results of the catalytic testing are described below. Examples 1-3 and Comparative Example 1 are reported in FIG. 1 and Table 2. The normalized conversion of CO as a function of time on stream (TOS) was derived by normalizing the measured CO conversion by the maximum CO conversion observed at TOS close to zero. The steady state rate of CO conversion, C₂-C₄ olefin selectivity, and methane selectivity were obtained after 300 hours TOS, and the decay rate was obtained from linearization between 50 and 350 TOS. Specifically, FIG. 1 shows the normalized CO conversion as a function of TOS for Examples 1-3 and Comparative Example 1. FIG. 1A shows a Comparative Example 1 curve 110 and an Example 1 curve 112. FIG. 1B shows a Comparative Example 1 curve 114 and Example 2 curve 116. FIG. 1C shows an Example 1 curve 118 and an Example 3 curve 120.

TABLE 2

| | | | | Steady | | C₂-C₄ | |
|---|---|---|---|---|---|---|---|
| | | | | state rate | | olefin | Methane |
| | Ga, | La, | Calcination | (μmol | Decay | selectivity, | Selectivity, |
| Catalyst | wt. % | wt. % | Temp. ° C. | g⁻¹s⁻¹) | rate, h⁻¹ | Cmol % | Cmol % |
| Ex. 1 | 2.74 | 3.89 | 550 | 7.90 | 1.76 × 10⁻⁴ | 44.10 | 1.0 |
| Ex. 2 | 2.99 | 0 | 700 | 7.35 | 1.86 × 10⁻⁴ | 44.79 | 1.33 |
| Ex. 3 | 2.79 | 3.99 | 700 | 7.82 | 1.0 × 10⁻⁴ | 45.26 | 1.16 |
| Comp. | 2.90 | 0 | 550 | 8.21 | 2.57 × 10⁻⁴ | 44.70 | 1.2 |
| Ex. 1 | | | | | | | |

As demonstrated in FIG. 1A and Table 2, the addition of lanthanum in the metal oxide catalyst component resulted in a decrease of the decay rate from 2.57×10⁻⁴ h⁻¹ (Comparative Example 1) to 1.76×10⁻⁴ h⁻¹ (Example 1), while maintaining similar reaction rates and selectivity.

As demonstrated in FIG. 1B and Table 2, the increase in the calcination temperature of the metal oxide catalyst component from 550° C. to 700° C. resulted in a decrease of the decay rate from 2.57×10⁻⁴ h⁻¹ (Comparative Example 1) to 1.86×10⁻⁴ h⁻¹ (Example 2), while maintaining similar reaction rates and selectivity.

As demonstrated in FIG. 1C and Table 2, the increase in the calcination temperature of the metal oxide catalyst component comprising lanthanum from 550° C. to 700° C. resulted in a decrease of the decay rate from 1.76×10⁻⁴ h⁻¹ (Example 1) to 1.0×10⁻⁴ h⁻¹ (Example 3), while maintaining similar reaction rates and selectivity.

As demonstrated in Table 2, the increase in the calcination temperature of the metal oxide catalyst component and the incorporation of lanthanum resulted in a decrease of the decay rate from 2.57×10⁻⁴ h⁻¹ (Comparative Example 1) to 1.0×10⁻⁴ h⁻¹ (Example 3), while maintaining similar reaction rates and selectivity.

The comparison of Examples 1-3 and Comparative Example 1 shows the importance of the metal oxide catalyst component calcination temperature, the incorporation of lanthanum into the metal oxide catalyst component, or both, on the decay rate of the catalyst, and thus the stability of the catalyst during the conversion of syngas to C₂-C₄ olefins

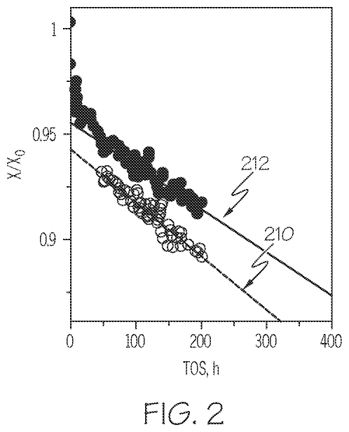

The results of the catalytic testing of Example 4 and Comparative Example 2 are reported in FIG. 2 and Table 3. The normalized conversion of CO as a function of time on stream (TOS) was measured, as described previously. The steady state rate of CO conversion, C₂-C₄ olefin selectivity, and methane selectivity were obtained at 40 hours TOS, and the decay rate was obtained from linearization between 40 and 200 TOS. Specifically, FIG. 2 shows the normalized CO conversion as a function of TOS for Examples 4 and Comparative Example 2. FIG. 2 shows Comparative Example 2 curve 210 and Example 4 curve 212.

TABLE 3

| | Metal oxide | | C₂-C₄ | | |
|---|---|---|---|---|---|
| | component | | olefin | Methane | |
| | calcination | Steady state | selec- | Selec- | |
| | Temperature | rate (μmol | tivity, | tivity, | Decay rate, |
| Catalyst | (° C.) | g⁻¹s⁻¹) | Cmol % | Cmol % | h⁻¹ |
| Ex. 4 | 700 | 6.85 | 49.8 | 1.1 | 2.13 × 10⁻⁴ |
| Comp. | 550 | 7.74 | 51.7 | 1.0 | 2.58 × 10⁻⁴ |
| Ex. 2 | | | | | |

As demonstrated in FIG. 2 and Table 3, the increase in the calcination temperature of the metal oxide catalyst component comprising lanthanum from 550° C. to 700° C. in the hybrid catalyst resulted in a decrease of the decay rate from 2.58×10⁻⁴ h⁻¹ (Comparative Example 2) to 2.13×10⁻⁴ h⁻¹ (Example 4), while maintaining similar reaction rates and selectivity. This comparison shows the importance of the metal oxide catalyst component calcination temperature on the decay rate of a hybrid catalyst comprising the metal oxide catalyst component, and thus the stability of the catalyst during the conversion of syngas to C₂-C₄ olefins.

### Example 7

A metal oxide catalyst component comprising gallium and dysprosium on a zirconia support was prepared by an incipient wetness impregnation method. An impregnation solution of gallium(III) nitrate hydrate (Ga(NO₃)₃·xH₂O) and dysprosium(III) nitrate hydrate (Dy(NO₃)₃·xH₂O) with a concentration of respectively 0.86 mol/L and 0.86 mol/L in DI water was prepared. 2 g of 60 mesh (250 μm) to 80 mesh (177 μm) ZrO₂ support (manufactured by NORPRO, product code SZ31164, BET surface area=100 m²/g, more than 95% monoclinic phase by XRD, pore volume=0.4 mL/g measured by DI water) was weighed and placed into a glass vial. After that, 0.8 mL of the Ga and Dy impregnation solution was added dropwise to the support while constantly shaking. After impregnation, the metal oxide catalyst component was dried at 80° C. in a forced convection oven overnight and calcined in a muffle furnace using the following program: from 25° C. to 550° C. at a heating rate of 3° C./min held at 550° C. for 4 hours. After calcination, the catalyst was re-sieved to 60-80 mesh.

### Example 8

A metal oxide catalyst component comprising gallium and samarium on a zirconia support was prepared by an incipient wetness impregnation method. An impregnation solution of gallium(III) nitrate hydrate (Ga(NO₃)₃·xH₂O) and samarium(III) nitrate hexahydrate (Sm(NO₃)₃·6H₂O) with a concentration of respectively 0.86 mol/L and 0.215 mol/L in DI water was prepared. 2 g of 40 mesh (250 μm) to 80 mesh (177 μm) ZrO₂ support (manufactured by NORPRO, product code SZ31164, BET surface area=100 m²/g, more than 95% monoclinic phase by XRD, pore volume=0.4 mL/g measured by DI water) was weighed and placed into a glass vial. After that, 0.8 mL of the Ga and Sm impregnation solution was added dropwise to the support while constantly shaking. After impregnation, the metal oxide catalyst component was dried at 80° C. in a forced convection oven overnight and calcined in a muffle furnace using the following program: from 25° C. to 550° C. at a heating rate of 3° C./min held at 550° C. for 4 hours. After calcination, the catalyst was re-sieved to 60-80 mesh.

### Example 9

A metal oxide catalyst component comprising gallium and gadolinium on a zirconia support was prepared by an incipient wetness impregnation method. An impregnation solution of gallium(III) nitrate hydrate (Ga(NO₃)₃·xH₂O) and gadolinium(III) nitrate hexahydrate (Gd(NO₃)₃·6H₂O) with a concentration of respectively 0.86 mol/L and 0.86 mol/L in DI water was prepared. 2 g of 40 mesh (250 μm) to 80 mesh (177 μm) ZrO₂ support (manufactured by NORPRO, product code SZ31164, BET surface area=100 m²/g, more than 95% monoclinic phase by XRD, pore volume=0.4 mL/g measured by DI water) was weighed and placed into a glass vial. After that, 0.8 mL of the Ga and Gd impregnation solution was added dropwise to the support while constantly shaking. After impregnation, the metal oxide catalyst component was dried at 80° C. in a forced convection oven overnight and calcined in a muffle furnace using the following program: from 25° C. to 550° C. at a heating rate of 3° C./min held at 550° C. for 4 hours. After calcination, the catalyst was re-sieved to 60-80 mesh.

### Comparative Example 3

A metal oxide catalyst component comprising gallium on a zirconia support was prepared by an incipient wetness impregnation method. An impregnation solution of gallium(III) nitrate hydrate (Ga(NO₃)₃·xH₂O) with a concentration of 0.86 mol/L in DI water was prepared. 2 g of 60 mesh (250 μm) to 80 mesh (177 μm) ZrO₂ support (manufactured by NORPRO, product code SZ31164, BET surface area=100 m²/g, more than 95% monoclinic phase by XRD, pore volume=0.4 mL/g measured by DI water) was weighed and placed into a glass vial. After that, 0.8 mL of the Ga impregnation solution was added dropwise to the support while constantly shaking. After impregnation, the metal oxide catalyst component was dried at 80° C. in a forced convection oven overnight and calcined in a muffle furnace using the following program: from 25° C. to 550° C. at a heating rate of 3° C./min held at 550° C. for 4 hours. After calcination, the catalyst was re-sieved to 60-80 mesh.

### Example 10—Catalytic Performance Data

The mixed metal oxide catalysts described in examples 7 to 9 and comparative example 3 were physically mixed with calcined SAPO-34 in 60-80 mesh sieve fraction in a 1:1 mass ratio prior to testing. Testing of the hybrid catalysts were performed in a quartz fixed bed reactor system (4 mm internal diameter) under the following conditions: 460° C., 40 bar, a weight hour space velocity (WHSV) of 3 h⁻¹, and a H₂/CO ratio of approximately 3.

Prior to contacting with syngas, the catalyst was heated under nitrogen (N₂) to reaction temperature and pressure. The reactor effluent composition was obtained by gas chromatography and the conversion and carbon based selectivities are calculated using equations (1) to (4). All data was collected under steady state conditions, after at least 40 hours time on stream.

TABLE 4

| | Steady State | C₂-C₄ | | |
|---|---|---|---|---|
| | CO | olefin | Methane | |
| | Conversion | selectivity, | Selectivity, | Normalized |
| Catalyst | (%) | Cmol % | Cmol % | Decay rate, h⁻¹ |
| Ex. 7 | 49.6 | 23.9 | 3.0 | 1.23 × 10⁻⁴ |
| Ex. 8 | 48.9 | 21.4 | 2.9 | 1.59 × 10⁻⁴ |
| Ex. 9 | 48.2 | 24.0 | 3.0 | 0.64 × 10⁻⁴ |
| Comp. Ex. 3 | 47.9 | 21.1 | 1.0 | 2.61 × 10⁻⁴ |

As demonstrated in Table 4, the addition of rare earth elements in the metal oxide catalyst component resulted in a decrease of the decay rate from 2.61×10⁻⁴ h⁻¹ (Comparative Example 3) to 0.64×10⁻⁴-1.59×10⁻⁴ (Example 7 to 9), while maintaining similar reaction rates and selectivity.

It is noted that one or more of the following claims utilize the term “where” or “in which” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.” For the purposes of defining the present technology, the transitional phrase “consisting of” may be introduced in the claims as a closed preamble term limiting the scope of the claims to the recited components or steps and any naturally occurring impurities. For the purposes of defining the present technology, the transitional phrase “consisting essentially of” may be introduced in the claims to limit the scope of one or more claims to the recited elements, components, materials, or method steps as well as any non-recited elements, components, materials, or method steps that do not materially affect the novel characteristics of the claimed subject matter. The transitional phrases “consisting of” and “consisting essentially of” may be interpreted to be subsets of the open-ended transitional phrases, such as “comprising” and “including,” such that any use of an open ended phrase to introduce a recitation of a series of elements, components, materials, or steps should be interpreted to also disclose recitation of the series of elements, components, materials, or steps using the closed terms “consisting of” and “consisting essentially of.” For example, the recitation of a composition “comprising” components A, B, and C should be interpreted as also disclosing a composition “consisting of” components A, B, and C as well as a composition “consisting essentially of” components A, B, and C. Any quantitative value expressed in the present application may be considered to include open-ended embodiments consistent with the transitional phrases “comprising” or “including” as well as closed or partially closed embodiments consistent with the transitional phrases “consisting of” and “consisting essentially of.”

As used in the Specification and appended Claims, the singular forms “a”, “an”, and “the” include plural references unless the context clearly indicates otherwise. The verb “comprises” and its conjugated forms should be interpreted as referring to elements, components or steps in a non-exclusive manner. The referenced elements, components or steps may be present, utilized or combined with other elements, components or steps not expressly referenced.

It should be understood that any two quantitative values assigned to a property may constitute a range of that property, and all combinations of ranges formed from all stated quantitative values of a given property are contemplated in this disclosure. The subject matter of the present disclosure has been described in detail and by reference to specific embodiments. It should be understood that any detailed description of a component or feature of one or more embodiments does not necessarily imply that the component or feature is essential to the particular embodiment or to any other embodiment. Further, it should be apparent to those skilled in the art that various modifications and variations can be made to the described embodiments without departing from the spirit and scope of the claimed subject matter.

1. A process for preparing C₂ to C₄ hydrocarbons comprising:
introducing a feed stream comprising hydrogen gas and a carbon-containing gas selected from the group consisting of carbon monoxide, carbon dioxide, and mixtures thereof into a reaction zone of a reactor; and
converting the feed stream into a product stream comprising C₂ to C₄ hydrocarbons in the reaction zone in the presence of a hybrid catalyst, wherein
the hybrid catalyst comprises:
a metal oxide catalyst component supported on zirconia, the metal oxide catalyst component comprising gallium oxide and a rare earth oxide; and
a microporous catalyst component comprising a molecular sieve having 8-MR (Member Ring) pore openings.

2. The process of claim 1, wherein the metal oxide catalyst component comprises greater than 1 wt. % and less than or equal to 7 wt. % of one or more rare earth elements.

3. The process of claim 1, wherein the metal oxide catalyst component comprises less than or equal to 10 wt. % of gallium.

4. The process of claim 1, wherein the metal oxide catalyst component comprises from 40.0 wt. % to 90.0 wt. % of the hybrid catalyst.

5. The process of claim 1, wherein the metal oxide catalyst component further comprises nickel, palladium or platinum.

6. The process of claim 1, wherein the rare earth oxide comprises samarium, gadolinium, dysprosium, lanthanum, cerium, neodymium, yttrium or combinations of two or more thereof.

7. The process of claim 1, wherein the microporous catalyst component comprises SAPO-34.

8. The process of claim 1, wherein the metal oxide catalyst component comprises less than 0.5 wt. % silicon and phosphorous.

9. The process of claim 1, wherein a temperature within the reaction zone during the converting is from 350° C. to 480° C.

10. The process of claim 1, wherein the C₂-C₄ hydrocarbons comprise 50 wt. % or greater C₂-C₄ olefins.

11. The process of claim 1, wherein the metal oxide catalyst component is calcined at a temperature that is from 400° C. to 800° C. to form a calcined metal oxide catalyst component before being mixed with the microporous catalyst component.

12. The process of claim 11, wherein the metal oxide catalyst component is calcined at a temperature that is from 550° C. to 700° C.

13. The process of claim 11, wherein the calcined metal oxide catalyst component is physically mixed with the microporous catalyst component.

14. The process of claim 11, wherein
the calcined metal oxide catalyst component and the microporous catalyst component are mixed with a binder to form a paste,
the paste is extruded to produce a hybrid catalyst,
the hybrid catalyst is dried and calcined, and
the binder is a colloidal solution, suspension, or gel of a binder precursor comprising oxides or hydroxides of aluminum, oxides or hydroxides of zirconium, or mixtures thereof.

15. The process of claim 14, wherein the binder comprises less than 0.5 wt. % silicon and phosphorus.
